(19) United States
(12) Patent Application Publication
(10) Pub. No.: US 2025/0276030 A1
(43) Pub. Date: Sep. 4, 2025

(54) EOM613 FOR TREATMENT OF COVID-19

(71) Applicant: EOM Pharmaceuticals Inc., Montvale, NJ (US)

(72) Inventors: Eli Goldberger, Monroe, NY (US); Shalom Z. Hirschman, Bronx, NY (US); Irach Taraporewala, White Plains, NY (US)

(73) Assignee: EOM Pharmaceuticals Inc., Montvale, NJ (US)

(21) Appl. No.: 18/032,986

(22) PCT Filed: Oct. 20, 2021
(86) PCT No.: PCT/US2021/055811

§ 371 Date: Apr. 20, 2023

Related U.S. Application Data
(60) Provisional application No. 63/094,172, filed on Oct. 20, 2020.
(60) Provisional application No. 63/094,168, filed on Oct. 20, 2020.

(51) Int. Cl. A61K 38/01; A61K 45/06; A61P 31/14

(52) U.S. Cl. CPC A61K 38/01; A61K 45/06; A61P 31/14

(57) ABSTRACT

Disclosed are methods of treating a subject with severe acute respiratory syndrome coronavirus 2 (SARS-COV-2) infection with EOM613. In several embodiments, a subject at risk of developing severe symptoms of SARS-COV-2 infection is selected for treatment. Also disclosed are methods wherein administering EOM613 reduces symptoms of SARS-COV-2 infection, including symptoms of cytokine storm syndrome or multisystem inflammatory syndrome.

### CROSS REFERENCE TO RELATED APPLICATIONS

This application is the § 371 U.S. National Stage of International Application No. PCT/US2021/055811, filed Oct. 20, 2021, which was published in English under PCT Article 21(2), which in turn claims the benefit of U.S. Provisional Application No. 63/094,168, filed Oct. 20, 2020, and U.S. Provisional Application No. 63/094,172, filed Oct. 20, 2020, which are both herein incorporated by reference in their entirety.

### FIELD

This relates to embodiments of a method of treating a subject with severe acute respiratory syndrome coronavirus 2 (SARS-COV-2) infection, as well as additional conditions such as cytokine storm syndrome, multisystem inflammatory syndrome, and Kawasaki disease.

### BACKGROUND

In 2019, a novel coronavirus (designated SARS-COV-2 by the World Health Organization) was identified as the causative agent of coronavirus disease 2019 (COVID-19). In March 2020, the World Health Organization (WHO) declared the COVID-19 outbreak a pandemic. Globally, SARS-COV-2 has caused more than 35.9 million COVID-19 cases and over one million fatalities as Oct. 6, 2020.

Although therapies with agents such as remdesevir, hydroxychloroquine, azithromycin, dexamethasone and interleukin-6 inhibitors have been used to treat patients with COVID-19, pulmonary and cardiac complications caused by cytokine release at sites of viral infection remain a leading cause of mortality in COVID-19 patients. The high case-fatality rate, vaguely defined epidemiology, and absence of prophylactic or therapeutic measures have created an urgent need for the development of effective treatments, particularly for patients experiencing or at risk of developing cytokine storm syndrome or multisystem inflammatory syndrome secondary to SARS-COV-2 infection.

### SUMMARY

Provided herein is a method of treating a subject with severe acute respiratory syndrome coronavirus 2 (SARS-COV-2) infection, comprising administering a therapeutically effective amount of EOM613 to the subject. In several embodiments, treating the subject with the therapeutically effective amount of EOM613 reduces at least one of the following symptoms of the SARS-COV-2 infection: respiratory distress, lung inflammation, hypoxia, myocarditis, renal disease, blood clotting, encephalitis, pneumonia, profound weakness, cytokine storm syndrome, and multisystem inflammatory syndrome.

In several embodiments, the subject is selected prior to treatment, for example by selecting a subject with signs or symptoms or a positive diagnosis of SARS-COV-2 infection, or a subject at risk of SARS-COV-2 infection, such as a subject with known exposure to SARS-COV-2 infection. In several embodiments, the subject with the SARS-COV-2 infection has or is at risk of COVID-19.

In some embodiments, administering the therapeutically effective amount of EOM613 to the subject comprises administering about 0.5 microliters to about 100 microliters EOM613 per kilogram of body weight per day to the subject. In some embodiments, administering the therapeutically effective amount of EOM613 to the subject comprises administering about 1 to about 2 ml EOM613 twice per day to the subject for 2-3 days followed by administering about 1 ml EOM613 once per day to the subject for 6-12 days. In some embodiments, administering the therapeutically effective amount of EOM613 to the subject comprises administering about 2 ml EOM613 subcutaneously twice per day for 3 days followed by 1 ml EOM613 subcutaneously twice per day for 7 days. In some embodiments, administering the therapeutically effective amount of EOM613 to the subject comprises administering about 2 ml EOM613 subcutaneously twice per day for 2-5 days followed by 2 ml EOM613 subcutaneously once per day for 3-5 days.

The foregoing and other features and advantages of this disclosure will become more apparent from the following detailed description of several embodiments, which proceeds with reference to the accompanying figures.

### BRIEF DESCRIPTION OF THE FIGURES

FIG. 1: Plasma IL-6 concentration in a COVID19 patient treated with EOM613.

FIG. 2: Lymphocyte count (%) in a COVID19 patient treated with EOM613.

FIG. 3: C-reactive protein (CRP) concentration in COVID19 patient treated with EOM613.

### DETAILED DESCRIPTION

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of an agent, such as EOM613, into a subject by a chosen route. Administration can be local or systemic. Exemplary routes of administration include, but are not limited to, oral, parenteral (such as subcutaneous, intramuscular, intradermal, intraperitoneal, and intravenous), sublingual, rectal, transdermal (for example, topical), intranasal, vaginal, and inhalation routes.

Control: A reference standard. In some embodiments, the control is a negative control sample obtained from a healthy patient. In other embodiments, the control is a positive control sample obtained from a patient diagnosed with a disease or condition, such as a SARS-COV-2 infection. In still other embodiments, the control is a historical control or standard reference value or range of values (such as a previously tested control sample, such as a group of patients diagnosed with a disease or condition, for example SARS-COV-2 infection, that have a known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus: A family of positive-sense, single-stranded RNA viruses that are known to cause severe respiratory illness. Viruses currently known to infect human from the coronavirus family are from the alphacoronavirus and betacoronavirus genera.

Non-limiting examples of betacoronaviruses include SARS-COV-2, Middle East respiratory syndrome coronavirus (MERS-COV), Severe Acute Respiratory Syndrome coronavirus (SARS-COV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV).

The viral genome is capped, polyadenylated, and covered with nucleocapsid proteins. The coronavirus virion includes a viral envelope containing type I fusion glycoproteins referred to as the spike(S) protein. Most coronaviruses have a common genome organization with the replicase gene included in the 5′-portion of the genome, and structural genes included in the 3′-portion of the genome.

Coronavirus Disease 2019 (COVID-19): A disease caused by SARS-COV-2 infection. Common symptoms include fever, cough, fatigue, shortness of breath or breathing difficulties, and loss of smell and taste. The incubation period may range from one to fourteen days. While most patients have mild symptoms, some develop acute respiratory distress syndrome (ARDS) possibly precipitated by cytokine storm, multi-organ failure, septic shock, and blood clots.

A host of underlying medical conditions are known to lead to increased risk of COVID-19, and severe symptoms of COVID-19, following infection with SARS-COV-2. Non-limiting examples include heart disease, cancer, chronic obstructive pulmonary disease, type 2 diabetes, type 1 diabetes, obesity, chronic kidney disease, sickle cell disease, asthma, liver disease, chronic lung disease, high blood pressure, or a suppressed immune system due to medical treatment, infection with a pathogen other than SARS-COV-2, or an autoimmune disorder.

The World Health Organization (WHO) has published testing guidelines for COVID-19 diagnosis (see, e.g., Laboratory Guidelines for the Detection and Diagnosis of COVID-19 virus infection, July 2020). The standard method of testing for SARS-COV-2 infection is real-time reverse transcription polymerase chain reaction (rRT-PCR) on respiratory samples obtained by a nasopharyngeal swab. Standard diagnostic methods of the detection of symptoms of COVID-19 is also utilized (e.g., lung inflammation, shortness of breath, low oxygen saturation, etc.

Cytokine Storm Syndrome: A severe immune reaction in which the innate immune system causes uncontrolled and excessive release of cytokines into the blood. Excessive production of proinflammatory cytokines can aggravate existing respiratory distress, as well as cause overwhelming systemic inflammation, hemodynamic instability, multiple organ dysfunction, and potentially death. Cytokine storm syndrome is also called hypercytokinemia. Detection of cytokine storm syndrome in a patient can be accomplished using standard diagnostic methods, including but not limited to elevation of plasma C-reactive protein (CRP) levels, elevation of interleukin-6 (IL-6) levels, abnormalities of markers of blood clotting such as D-dimer or fibrinogen and elevated ferritin levels. Protocols for detecting cytokine storm in a COVID-19 patient are known and described, for example, in Soy et al., Clin Rheumatol., 39(7):2085-2094, 2020. Additional information concerning cytokine storm syndrome can be found, for example, in Ye et al., Journal of Infection, 80(6):607-613, 2020.

Detecting: To identify the existence, presence, or fact of something.

Inhibiting or Treating a Disease or Condition: Inhibiting the full development of a disease or condition, for example, in a subject who has or is at risk for a condition such as cytokine storm syndrome or multisystem inflammatory syndrome in a subject who is at risk for developing secondary complications of a viral infection, such as SARS-COV-2 infection. “Treatment” refers to a therapeutic intervention that ameliorates a sign or symptom of a condition after the condition has begun to develop. The term “ameliorating,” with reference to a disease, syndrome, or pathological condition, refers to any observable beneficial effect of the treatment. The beneficial effect can be evidenced, for example, by a delayed onset of clinical symptoms in a susceptible subject, a reduction in severity of some or all clinical symptoms, a slower progression, a reduction in the viral load, an improvement in the overall health or well-being of the subject, or by other parameters that are specific to the particular disease, syndrome, or other pathological condition. Inhibiting a disease or condition can include preventing or reducing the risk of the disease or condition, such as preventing or reducing the risk of severe symptoms (e.g., cytokine storm syndrome or multisystem inflammatory syndrome) due to viral infection such as SARS-COV-2 infection. A “prophylactic” treatment is a treatment administered to a subject who does not exhibit signs of a disease or exhibits only early signs for the purpose of decreasing the risk of developing pathology. In some embodiments, the disclosed methods are therapeutic and not prophylactic.

Kawasaki disease: An inflammatory disease of unknown origin that that results in a fever and mainly affects children under 5 years of age, and a leading cause of heart disease in children. In affected patients, blood vessels become inflamed throughout the body. The fever typically lasts for more than five days and is not treated with standard medication. Other common symptoms include large lymph nodes in the neck, a rash in the genital area, and red eyes, lips, palms, or soles of the feet. In some children, coronary artery aneurysms form in the heart leading to heart disease.

While the specific cause is unknown, it is thought to result from an excessive immune system response to an infection in children who are genetically predisposed. Diagnosis is usually based on a person's signs and symptoms. Other tests such as an ultrasound of the heart and blood tests may support the diagnosis. Detection of multisystem inflammatory syndrome in a patient can be accomplished using established diagnostic methods, including but not limited to persistent fever despite use of antipyretics, tachycardia and hypotension, leukocytosis with lymphocytopenia, elevated C-reactive protein, elevated D-dimer and B-type natriuretic peptide. Additional information concerning Kawasaki disease can be found, for example, in Hamden et al., British Medical Journal, 338:b1514, 1133-1138, 2009.

Lung inflammation: A localized protective response elicited by injury to lung tissue that serves to sequester the inflammatory agent. Lung inflammation is characterized by the appearance in or migration into the lung of any class of leukocyte in numbers that exceed the number of such cells found within such region of tissue under normal (healthy) circumstances. Detection and evaluation of lung inflammation in a subject can be accomplished by any appropriate means, including radiographic evaluation by X-ray. Lung inflammation can be classified as either acute or chronic. Acute lung inflammation is the initial response of the body to harmful stimuli and is achieved by the increased movement of plasma and leukocytes from the blood into the injured lung tissue. A cascade of biochemical events propagates and matures the inflammatory response, involving the local vascular system, the immune system, and various cells within the injured lung tissue.

Lymphocytopenia: A condition defined by an abnormally low lymphocyte count in the blood. The condition is also referred to as lymphocytic leukopenia and lymphopenia. Additional information concerning Lymphocytopenia in the context of COVID19 can be found, for example, in Wagner et al., Int. J Lab Hematol. 10.1111/ijlh.13288. 10 Jul. 2020, doi:10.1111/ijlh.13288, 338:b1514, 1133-1138, 2009.

Multisystem Inflammatory Syndrome: A systemic disorder involving persistent fever and severe inflammation across multiple body systems, in response to infection with a viral agent such as SARS-COV-2. Multisystem inflammatory syndrome is most often observed in children under the age of 5, although adults are also known to be affected. Along with persistent fever, the first symptoms often include acute abdominal pain with diarrhea or vomiting. Muscle pain and general tiredness are frequent, and low blood pressure is also common. Other symptoms sometimes include pink eye, rashes, enlarged lymph nodes, swollen hands and feet, and “strawberry tongue” (glossitis which manifests with enlarged fungiform papillae). The patient may develop cytokine storm syndrome. Heart failure is common, and inflammation of the heart muscle has been reported. Clinical complications can include damage to the heart muscle, respiratory distress, acute kidney injury, and increased blood coagulation. Coronary artery abnormalities can develop (ranging from dilatation to aneurysms). Detection of multisystem inflammatory syndrome in a patient can be accomplished using standard diagnostic methods. Additional description of multisystem inflammatory syndrome in children and adults can be found, for example, in Godfred-Cato et al., COVID-19-associated multisystem inflammatory syndrome in children—United States, March-July 2020. MMWR Morb Mortal Wkly Rep 2020;69:1074-80; and Morris et al. Case Series of Multisystem Inflammatory Syndrome in Adults Associated with SARS-COV-2 Infection—United Kingdom and United States, MMWR Morb Mortal Wkly Rep 2020;69:1450-1456.

Nucleotides: Organic molecules consisting of a nucleoside and a phosphate. A nucleotide refers to a ribonucleotide, deoxynucleotide or a modified form of either type of nucleotide. They serve as monomeric units of the nucleic acid polymers deoxyribonucleic acid and ribonucleic acid.

Nucleic Acid Molecule: A polymeric form of nucleotides, which may include both sense and anti-sense strands of RNA, cDNA, genomic DNA, and synthetic forms and mixed polymers of the above. The term “nucleic acid molecule” as used herein is synonymous with “nucleic acid” and “polynucleotide.” A nucleic acid molecule is usually at least 10 bases in length, unless otherwise specified. The term includes single-and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Normal level: a value that falls within the “normal” reference range (“normal range”) of a population that is used by health care professionals to interpret medical tests, such as blood sample tests. In some embodiments, a subject has an elevated level of one or more of D-dimer, troponin, C-reactive protein, procalcitonin, or cytokine, and administering the therapeutically effective amount of EOM613 to the subject decreases the elevated level to a normal level.

Pharmaceutically Acceptable Carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents, a syringe for injection into a subject, lyophilized for subsequent solubilization and administration, in a solid or controlled release dosage, or in an oral lozenge form.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Pulmonary function: The function of the respiratory system, which can be measured through a variety of tests, including, but not limited to measurements of airflow (e.g. spirometry) or arterial blood gases (for example, oxygen saturation level, such as SpO2). Measurements of airflow included airflow rate, peak expiratory flow rate (PEFR), forced expiratory volume in the first second (FEV₁), and maximal midexpiratory rate (MMEFR).

SARS-COV-2: Also known as Wuhan coronavirus or 2019 novel coronavirus, SARS-COV-2 a positive-sense, single stranded RNA virus of the genus betacoronavirus that has emerged as a highly fatal cause of severe acute respiratory infection. The viral genome is capped, polyadenylated, and covered with nucleocapsid proteins. The SARS-COV-2 virion includes a viral envelope with large spike glycoproteins. The SARS-COV-2 genome, like most coronaviruses, has a common genome organization with the replicase gene included in the 5′-two thirds of the genome, and structural genes included in the 3′-third of the genome. The SARS-COV-2 genome encodes the canonical set of structural protein genes in the order 5′—spike(S)—envelope (E)—membrane (M) and nucleocapsid (N)—3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days. The disease resulting from a SARS-COV-2 infection is called COVID-19.

Standard methods for detecting viral infection may be used to detect SARS-COV-2infection, including but not limited to, assessment of patient symptoms and background and genetic tests such as reverse transcription-polymerase chain reaction (rRT-PCR). The test can be done on patient samples such as respiratory or blood samples.

Subject: Living multi-cellular vertebrate organisms, a category that includes human and non-human mammals, such as non-human primates, pigs, camels, bats, sheep, cows, dogs, cats, rodents, and the like. In an example, a subject is a human. In an additional example, a subject is selected that is in need of inhibiting a SARS-COV-2 infection. For example, the subject is either uninfected and at risk of the SARS-COV-2 infection or is infected and in need of treatment.

Therapeutically Effective Amount: The amount of agent, such as a pharmaceutical composition, that is sufficient to prevent (including prophylaxis), treat, reduce, and/or ameliorate the symptoms and/or underlying cause of a disease, syndrome, or other pathological condition. For example, to prevent, inhibit, and/or treat SARS-COV-2 infection and/or disease (such as COVID-19) or secondary syndromes (such as cytokine storm syndrome) resulting therefrom. In some embodiments, a therapeutically effective amount is sufficient to reduce or eliminate a symptom of a disease. For instance, this can be the amount necessary to inhibit or prevent pathogen replication or to measurably alter outward symptoms of pathogen infection.

World Health Organization (WHO) Ordinal Scale for Clinical Improvement: A scale developed by the World Health Organization used as a reference to categorize COVID-19 disease severity into eight ordered scores (see, Table 2). The WHO Ordinal Scale for Clinical Improvement may also be referred to as the “WHO COVID-19 Symptom Severity Scale.” A “WHO score” as used in this application refers to a score on the WHO Ordinal

Scale for Clinical Improvement. Additional description and application of the WHO Ordinal Scale for Clinical Improvement can be found, for example, in: WHO R&D Blueprint, Novel Coronavirus, COVID-19 Therapeutic Trial Synopsis, World Health Organization, Feb. 18, 2020, herein incorporated by reference in its entirety.

It is understood that a therapeutically effective amount encompasses a fractional dose that contributes in combination with previous or subsequent administrations to attaining a desired response. For example, a therapeutically effective amount of an agent can be administered in a single dose, or in several doses, for example daily, during a course of treatment. However, the therapeutically effective amount and timing of administration can depend on the subject being treated, the severity and type of the condition being treated, and the manner of administration. A therapeutically effective amount can be determined by varying the dosage and measuring the resulting response, such as, for example, a reduction in pathogen titer. Effective amounts also can be determined through various in vitro, in vivo or in situ assays. A unit dosage form of the agent can be packaged in a therapeutic amount, or in multiples of the therapeutic amount, for example, in a vial (such as with a pierceable lid) or syringe having sterile components.

### II. EOM613

EOM613, also known as Product R or AVR118, is a composition comprising nucleotides and peptides having molecular weights of not more than 14 KDa, primarily not more than 8 KDa. The nucleotides and peptides are breakdown products of casein, peptone, RNA and serum albumin. EOM613 and its production are described, for example, in U.S. Pat. Nos. 6,528,098, 6,921,542, 7,074,767, 7,524,661, and 8,084,239, which are incorporated by reference herein in their entirety. In these patent documents, EOM613 is referred to as Product R. A brief description of EOM613 and its production is provided herein.

Sixteen constituent compounds have been identified and characterized in EOM613: three nucleosides, two nucleoside diphosphates and eight nucleoside monophosphates, together with two peptides (one of them a peptide-nucleic acid conjugate) and sodium chloride (resulting from the neutralization of sodium hydroxide with hydrochloric acid during the manufacturing process).

The longer peptide (referred to as “peptide-A”) is 31-amino-acid peptide derived from bovine beta-casein with a molecular weight of 3536.24 Da. The shorter peptide (referred to as “peptide-B) is a peptide-oligonucleotide conjugate including a 21-amino acid long peptide attached at a seine residue at position 18 to a diadenine (3′-5′) diribonucleotide through a diphosphodiester linkage at the 3′-position. This nucleopeptide conjugate has a MW 2215 peptide moiety attached to a 740 MW nonpeptidic adduct to give a total MW of 2955. Peptide-A and peptide-B are present in EOM613 in approximately equal amount by weight. The amount of peptide-A and peptide-B in EOM613 is about 4.4-7.0 mg/mL, preferably 4.8-5.3 mg/mL, as determined by a Lowry protein assay.

Generally, EOM613 is prepared according to the following manner: casein, beef peptone, RNA, BSA, and sodium hydroxide are suspended in proportions of, by weight, about 35-50% casein, about 15-40% beef peptone, about 10-25% RNA, about 1-10% BSA, and about 5-25% sodium hydroxide in an appropriate volume of distilled water. Any RNA source may be used, such as plant RNA or yeast RNA. In several embodiments, yeast RNA is used as the RNA source for generating the EOM613 composition. The ratio of total protein to the volume of distilled water is about 1.5-2.5 to about 100 by weight, preferably about 2.2 to about 100 by weight. Thus, every 1.5-2.5 grams of the total protein are suspended in about 100 milliliters of distilled water. Any suitable source may be used to obtain the starting materials. The starting materials are available commercially or can be readily prepared by a person of ordinary skill in the art.

The suspension as prepared above is then autoclaved at a pressure of approximately 5-15 pounds per square inch, for example 8-10 pounds per square inch, under an elevated temperature in a range, for example, of from about 150°-300° F. (such as about 200°-230° F.), over a period of approximately 2-10 hours, typically more than 3 hours. As known to a person of ordinary skill in the art, under such conditions RNA may be completely hydrolyzed into nucleotides. After autoclaving, the solution is cooled to room temperature, and then allowed to stay at a temperature of 3° to 8° C. for at least 12 hours to precipitate insoluble elements. Alternatively, the cooled solution may be centrifuged at a temperature below 8° C. to remove the insoluble elements.

The resulting solution is then filtered through a 2 micron and a 0.45 micron filters under an inert gas such as nitrogen or argon at a pressure of about 1-6 psi. In a similar manner the solution is filtered again through a pyrogen retention filter, such as a pyrogen retention filter of about 0.2 micron. After the filtration, the solution optionally may be cooled at 3 to 8° C. again for at least about 12 hours and filtered again in the same way as described above.

The resulting filtrate is then assayed for total nitrogen content using any suitable method, such as the Kjeldahl method (see Kjeldahl, Z. Anal. Chem., Vol. 22, p366, 1883), and related methods based on the Kjeldahl method. Based on the assay, the filtrate is then diluted with chilled distilled water to an appropriate volume having a preferred total nitrogen content ranging from about 165 to about 210 mg/ml. The pH of the diluted solution is then adjusted with HCl to a physiological pH of about 7.3 to about 7.6, after which the diluted solution is filtered again through a 0.2 micron filter under an inert gas as described above. The final filtrate has a light absorption spectrum with typical absorption ratios of 2.0 (±10%) at 260 nm/280 nm and 1.4 (±10%) at 260 nm/230 nm.

The use of filtration discussed above is to remove bacteria or other particles having similar size to or larger size than bacteria. Any filter regardless its manufacturer or material from which it is made that is suitable for this purpose may be used in the production of EOM613.

The final filtrate is then filled and sealed into appropriate vials, such as 2 ml or 10 ml glass vials under an inert gas. The filled vials are autoclaved for final sterilization, after which they are ready for use. Unless otherwise specified, the final filtrate is in the concentration of EOM613 suitable for administration to a subject. Thus, after following the proceeding steps, the EOM613 composition is ready to administer to a subject.

### III. Methods of Treatment Using EOM613

Methods of treating certain diseases and conditions by administering a therapeutically effective amount of EOM613 to a subject in need thereof are provided herein.

In some embodiments, the therapeutically effective amount of EOM613 is administered to a subject to inhibit or treat SARS-COV-2 infection in the subject. Administration of the therapeutically effective amount of EOM613 to the subject reduces and/or eliminates one or more symptoms of the SARS-COV-2 infection in the subject.

In some embodiments, a subject with or at risk of SARS-COV-2 infection is selected for treatment with EOM613. In some embodiments, the subject selected for treatment has COVID-19. In some embodiments, the subject selected for treatment has a SARS-COV-2 infection and also one or more of the following underlying medical conditions: heart disease, cancer, chronic obstructive pulmonary disease, type 2 diabetes, type 1 diabetes, obesity, chronic kidney disease, sickle cell disease, asthma, liver disease, chronic lung disease, high blood pressure, or a suppressed immune system due to medical treatment, infection with a pathogen other than SARS-COV-2, or an autoimmune disorder. The subject may be any age, such as at least 75 years old.

Subjects infected with SARS-COV-2 may display a range of symptoms, from mild to severe, or may have no symptoms at all. In one example, the selected subject has no overt symptoms of SARS-COV-2 infection and treatment with EOM613 is prophylactic to inhibit further development of disease or symptoms. In other examples, the selected subject has one or more symptoms of SARS-COV-2 infection and treatment with EOM613 is a therapeutic treatment. Symptoms of SARS-COV-2 infection include, but are not limited to: fever, cough, fatigue, respiratory distress, loss of taste or smell, muscle aches, chills, sore throat, runny nose, headache, chest pain, conjunctivitis, hypoxia, myocarditis, renal disease, blood clotting, encephalitis, pneumonia, profound weakness, cytokine storm syndrome, and multisystem inflammatory syndrome. In a specific example, the selected subject has one or more of the following symptoms of SARS-COV-2 infection: respiratory distress, hypoxia, myocarditis, renal disease, blood clotting, encephalitis, pneumonia, profound weakness, cytokine storm syndrome, and multisystem inflammatory syndrome. In a preferred embodiment, the selected subject has cytokine storm syndrome or multisystem inflammatory syndrome.

Treatment with EOM613 reduces one or more of the symptoms of the SARS-COV-2 infection. The symptoms of the SARS-COV-2 infection do not need to be eliminated for the method to be effective. For example, the method can reduce one or more symptoms of the SARS-COV-2 infection by at least 10%, at least 20%, at least 50%, at least 60%, at least 70%, at least 80%, at least 90%, at least 95%, at least 98%, or even at least 100% (elimination of the symptom), as compared to a suitable control.

In some embodiments, the method results in a reduction of lung inflammation in the subject. In some embodiments, the method results in a reduction in IL-6 levels in the subject to a normal range. In some embodiments, the method results in an increase in pulmonary function in the subject, such as an increase in airflow rate, peak expiratory flow rate (PEFR), forced expiratory volume in the first second (FEV₁), and maximal midexpiratory rate (MMEFR). In some embodiments, the method results in an increase in blood oxygen saturation level in the subject.

In some embodiments, the subject has an elevated level of one or more of D-dimer, troponin (e.g., troponin-I, troponin-T, and troponin-C), C-reactive protein, procalcitonin, and cytokine (e.g., interleukin-6 (IL-6), interleukin-12 (IL-12), interleukin-10 (IL-10), interleukin-2 (IL-2), tumor necrosis factor alpha (TNF-α), or interferon gamma (INF-γ)). In some examples, the troponin is troponin-I. In some examples, the cytokine is an interleukin, for example, one or more of interleukin-6 (IL-6), interleukin-12 (IL-12), interleukin-10 (IL-10), and interleukin-2 (IL-2). In some embodiments, the method results in a reduction of one or more of elevated D-dimer, troponin (e.g., troponin-I), C-reactive protein, procalcitonin, and cytokine (e.g., IL-6) levels. D-dimer, troponin, C-reactive protein, procalcitonin, or cytokines are measured from a sample obtained from the patient, such as a tissue or bodily fluid. In some examples, D-dimer, troponin, C-reactive protein, procalcitonin, or cytokines are measured from a blood sample from the subject. The blood sample can be whole blood, or a blood derivative, such as plasma or serum.

In some embodiments, administering EOM613 decreases the level of one or more of D-dimer, troponin (e.g., troponin-I, troponin-T, and troponin-C), C-reactive protein, procalcitonin, or cytokine (e.g., interleukin-6 (IL-6), interleukin-12 (IL-12), interleukin-10 (IL-10), interleukin-2 (IL-2), tumor necrosis factor alpha (TNF-α), or interferon gamma (INF-γ)) levels in the subject, for example, the method can decrease the level by at least 10%, at least 20%, at least 30%, at least 40%, at least 50%, at least 60%, at least 70%, at least 80%, at least 90%, at least 95%, at least 98%, or even at least 100%, as compared to a suitable control (e.g., relative to the level prior to treatment). In some examples, the troponin is troponin-I. In some examples, the cytokine is an interleukin, for example, one or more of interleukin-6 (IL-6), interleukin-12 (IL-12), interleukin-10 (IL-10), or interleukin-2 (IL-2). In some examples, administering the therapeutically effective amount of EOM613 to the subject decreases the level of one or more of D-dimer, troponin, C-reactive protein, procalcitonin, or cytokine level, to a non-detectable or a normal level.

D-dimer, troponin (e.g., troponin-I, troponin-T, and troponin-C), C-reactive protein, procalcitonin, or cytokine (e.g., interleukin-6 (IL-6), interleukin-12 (IL-12), interleukin-10 (IL-10), interleukin-2 (IL-2), tumor necrosis factor alpha (TNF-α), or interferon gamma (INF-γ)) levels can be periodically measured and/or compared throughout a course of treatment, for example, every day, every other day, once every three days, weekly, or monthly. In some examples, D-dimer, troponin, C-reactive protein, procalcitonin, or cytokine levels are measured prior to treatment, and/or on about day 1 of treatment, about day 2 of treatment, about day 3 of treatment, about day 4 of treatment, about day 5 of treatment, about day 6 of treatment, about day 7 of treatment, about day 8 of treatment, about day 9 of treatment, about day 10 of treatment, about day 11 of treatment, about day 12 of treatment, about day 13 of treatment, about day 14 of treatment, about day 15 of treatment, about day 16 of treatment, about day 17 of treatment, about day 18 of treatment, about day 19 of treatment, about day 20 of treatment, about day 24 of treatment, about day 26 of treatment, about day 28 of treatment, about day 30 of treatment, about day 35 of treatment, about day 40 of treatment, about day 45 of treatment, about day 50 of treatment, about day 55 of treatment, about day 60 of treatment, or any combination thereof. In some examples, D-dimer, troponin, C-reactive protein, procalcitonin, or cytokine levels are measured prior to treatment (or early in treatment, such as about day 1) and are compared to respective levels after one or more days of EOM613 treatment, for example, after about 1 day of treatment, about 2 days of treatment, about 3 days of treatment, about 4 days of treatment, about 5 days of treatment, about 6 days of treatment, about 7 days of treatment, about 8 days of treatment, about 9 days of treatment, about 10 days of treatment, about 12 days of treatment, about 14 days of treatment, about 16 days of treatment, about 18 days of treatment, about 20 days of treatment, about 24 days of treatment, about 28 days of treatment, about 30 days of treatment, about 35 days of treatment, about 40 days of treatment, about 45 days of treatment, about 50 days of treatment, about 55 days of treatment, about 60 days of treatment, or longer. In some examples, D-dimer, troponin, C-reactive protein, procalcitonin, or cytokine levels are measured in the subject prior to EOM613 treatment (or early in treatment, such as about day 1) and are compared to respective levels after completing treatment with EOM 613.

In some embodiments, the method results in a reduction of one or more of elevated D-dimer levels, troponin (e.g., troponin-I, troponin-T, or troponin-C) levels, C-reactive protein levels, procalcitonin, and IL-6 levels, and an increase in oxygenation levels (for example, increased Sp02 measurements) compared to before the treatment with a therapeutically effective amount of EOM613. In some embodiments, the method results in a reduction of elevated D-dimer levels, troponin levels, C-reactive protein levels, and IL-6 levels, and increase in oxygenation levels (for example, increased SpO2 measurements) compared to before the treatment with a therapeutically effective amount of EOM613. In some embodiments, the method results in increased lymphocyte count of the subject compared to before the treatment with a therapeutically effective amount of EOM613.

In some embodiments, the subject is diagnosed or presumed to have COVID-19. COVID-19 severity can be categorized using the World Health Organization (WHO) Ordinal Scale for Clinical Improvement. In some examples, the subject has a score of at least 1, at least 2, at least 3, at least 4, at least 5, at least 6, or at least 7 on the WHO Ordinal Scale for Clinical Improvement prior to initiation of treatment with EOM613. In some examples, the subject has a score of about 1 to about 7, about 1 to about 6, about 1 to about 5, about 1 to about 4, about 1 to about 3, about 1 to about 2, about 2 to about 7, about 3 to about 7, about 4 to about 7, about 5 to about 7, about 6 to about 7, about 2 to about 6, about 2 to about 5, about 2 to about 4, about 2 to about 3, about 3 to about 6, about 3 to about 5, about 3 to about 4, about 4 to about 6, about 4 to about 5, or about 5 to about 6, prior to initiation of treatment with EOM613. In some examples, the subject has a score of 4 or higher on the WHO Ordinal Scale for Clinical Improvement prior to initiation of treatment. In some examples, the subject has a score of 5 or higher on the WHO Ordinal Scale for Clinical Improvement prior to initiation of treatment. In some examples, the subject has a score of 6 or higher on the WHO Ordinal Scale for Clinical Improvement prior to initiation of treatment. In some examples, EOM 613 treatment reduces the score of a subject on the WHO Ordinal Scale for Clinical Improvement, for example, reduces the score by at least 1, at least 2, at least 3, at least 4, at least 5, at least 6, or at least 7. In some examples, EOM 613 treatment reduces the score of a subject on the WHO Ordinal Scale for Clinical Improvement to less than 4, for example, reduces the score to at least 0, 1, 2, or 3.

In some embodiments, administering EOM613 is effective to inhibit SARS-COV-2-induced cytokine storm syndrome. In some embodiments, the treatment reduces or maintains cytokine production or accumulation in the subject, for example, to a normal level. In some embodiments, EOM613 is administered as a prophylactic measure in an amount effective to inhibit onset of cytokine storm syndrome elicited by SARS-COV-2 infection. Inhibiting the onset of cytokine storm syndrome in the subject does not require complete inhibition, but instead may be a partial inhibition or reduction in symptoms associated with cytokine storm syndrome.

In some embodiments, administering EOM613 is effective to inhibit SARS-COV-2 induced multisystem inflammatory syndrome. In some such embodiments, the amount of EOM613 administered to the subject is effective to reduce organ inflammation or damage in the subject due to the multisystem inflammatory syndrome. In specific non-limiting examples, the reduced organ damage is reduced damage to the heart, kidneys, and/or lungs. In some embodiments the subject administered EOM613 in an amount effective to inhibit SARS-COV-2 induced multisystem inflammatory syndrome, or to reduce organ damage or inflammation, is a child, such as a child 5 years old or younger.

In some embodiments, administering EOM613 is effective to inhibit SARS-COV-2-induced Kawasaki disease. In some such embodiments, the amount of EOM613 administered to the subject is effective to reduce cardiac damage in the subject due to the Kawasaki disease. In some embodiments, EOM613 is administered as a prophylactic measure in an amount effective to inhibit onset of Kawasaki disease elicited by SARS-COV-2 infection.

In some embodiments, the therapeutically effective amount of EOM613 is administered to a subject to inhibit or treat cytokine storm syndrome in the subject. Administration of the therapeutically effective amount of EOM613 to the subject reduces and/or eliminates one or more symptoms of the cytokine storm syndrome in the subject. In some embodiments, a subject with or at risk of cytokine storm syndrome is selected for treatment with EOM613.

Treatment with EOM613 reduces one or more of the symptoms of the cytokine storm syndrome. The symptoms of the cytokine storm syndrome do not need to be eliminated for the method to be effective. For example, the method can reduce one or more symptoms of the cytokine storm syndrome by at least 10%, at least 20%, at least 50%, at least 60%, at least 70%, at least 80%, at least 90%, at least 95%, at least 98%, or even at least 100% (elimination of the symptom), as compared to a suitable control. In some embodiments, the treatment of a subject with cytokine storm syndrome with the therapeutically effective amount of EOM613 results in a reduction of one or more of elevated D-dimer levels, troponin (e.g., troponin-I, troponin-T, and troponin-C) levels, C-reactive protein levels, and IL-6 levels, and increase in oxygenation levels (for example, increased SpO2 measurements) compared to before the treatment with a therapeutically effective amount of EOM613.

In some embodiments, the therapeutically effective amount of EOM613 is administered to a subject to inhibit or treat multisystem inflammatory syndrome in the subject. Administration of the therapeutically effective amount of EOM613 to the subject reduces and/or eliminates one or more symptoms of the multisystem inflammatory syndrome in the subject. In some embodiments, a subject with or at risk of multisystem inflammatory syndrome is selected for treatment with EOM613.

Treatment with EOM613 reduces one or more of the symptoms of the multisystem inflammatory syndrome. The symptoms of the multisystem inflammatory syndrome do not need to be eliminated for the method to be effective. For example, the method can reduce one or more symptoms of the multisystem inflammatory syndrome by at least 10%, at least 20%, at least 50%, at least 60%, at least 70%, at least 80%, at least 90%, at least 95%, at least 98%, or even at least 100% (elimination of the symptom), as compared to a suitable control. In some embodiments, the treatment of a subject with multisystem inflammatory syndrome with the therapeutically effective amount of EOM613 results in resolution of leukocytosis or lymphocytopenia, increased band neutrophils, a reduction in elevated plasma levels of: hepatic enzymes (such as aspartate aminotransferase (AST) and alanine aminotransferase (ALT)), erythrocyte sedimentation rate (ESR), Ferritin, C-reactive protein (CRP), and/or Interleukin-6 (IL-6). In some embodiments, the treatment of a subject with multisystem inflammatory syndrome with the therapeutically effective amount of EOM613 results in increased lymphocyte count of the subject compared to before the treatment with a therapeutically effective amount of EOM613.

In some embodiments, the therapeutically effective amount of EOM613 is administered to a subject to inhibit or treat Kawasaki disease in the subject. Administration of the therapeutically effective amount of EOM613 to the subject reduces and/or eliminates one or more symptoms of the Kawasaki disease in the subject. In some embodiments, a subject with or at risk of multisystem inflammatory syndrome is selected for treatment with EOM613.

Treatment with EOM613 reduces one or more of the symptoms of the Kawasaki disease. The symptoms of the Kawasaki disease do not need to be eliminated for the method to be effective. For example, the method can reduce one or more symptoms of the Kawasaki disease by at least 10%, at least 20%, at least 50%, at least 60%, at least 70%, at least 80%, at least 90%, at least 95%, at least 98%, or even at least 100% (elimination of the symptom), as compared to a suitable control. In some embodiments, the treatment of a subject with Kawasaki disease with the therapeutically effective amount of EOM613 results in a reduction of rash, erythema, and lip swelling, resolution of leukocytosis or lymphocytopenia, increased band neutrophils, a reduction in elevated plasma levels of: hepatic enzymes (such as aspartate aminotransferase (AST) and alanine aminotransferase (ALT)), erythrocyte sedimentation rate (ESR), Ferritin, C-reactive protein (CRP), and/or Interleukin-6 (IL-6). In some embodiments, the treatment of a subject with Kawasaki disease with the therapeutically effective amount of EOM613 results in increased lymphocyte count of the subject compared to before the treatment with a therapeutically effective amount of EOM613. In some embodiments, the treatment of a subject with Kawasaki disease with the therapeutically effective amount of EOM613 results in resolution of lymphocytopenia.

In some embodiments, the therapeutically effective amount of EOM613 is administered to a subject to inhibit or treat lymphocytopenia in the subject. Administration of the therapeutically effective amount of EOM613 to the subject results in an increase in the lymphocyte count towards a normal level. Lymphocyte count is often measured as a percentage of white blood cells in a subject. A normal range of lymphocytes is from 20-40% of white blood cells. In several embodiments, treatment of a subject with lymphocytopenia (e.g., a subject with a lymphocyte percentage below 10%) results in an increase in the lymphocyte percentage to a normal lymphocyte percentage range of from 20-40%. In some embodiments, a subject with or at risk of lymphocytopenia is selected for treatment with EOM613.

The dose of EOM613 administered to the subject in the methods provided herein may vary depending on multiple factors, including the amount and timing of administration, the subject being treated, the severity and type of the condition being treated, and the manner of administration. Generally, an appropriate dosage is used that is sufficient to alleviate symptoms of disease without causing unwanted side effects. In some embodiments, about 0.5 microliters to about 100 microliters (such as about 2.5 microliters to about 40 microliters, or about 10 microliters to about 25 microliters) EOM613 per kilogram of body weight per day is administered to the subject. In some embodiments, about 1-2 mL (such as 1 or 2 mL) EOM613 is administered to the subject per day. The desired dose may be administered once a day, or in two, three or more sub-doses at appropriate intervals, generally equally spread in time, throughout the day. In some embodiments, EOM613 is administered for about 1 day, about 2 days, about 3 days, about 4 days, about 5 days, about 6 days, about 7 days, about 8 days, about 9 days, about 10 days, about 11 days, about 12 days, about 14 days, about 16 days, about 18 days, about 20 days, about 22 days, about 24 days, about 28 days, or longer. In some embodiments, about 1 to about 2 ml (such as 1 or 2 ml) EOM613 is administered twice per day to the subject. In some embodiments, about 2 ml EOM613 is administered twice per day to the subject for two days, and then about 1 ml EOM613 is administered twice per day to the subject for eight days. In some embodiments, about 2 ml EOM613 is administered twice per day to the subject for three days, and then about 1 ml EOM613 is administered twice per day to the subject for seven days. In some embodiments, about 2 mL EOM613 is administered twice per day to the subject for two days, and then about 2 mL EOM613 is administered once daily for three days. In some examples, about 1 to about 2 ml EOM613 is administered for two to five days, subsequently followed by administering about 1 to about 2 ml EOM613 once per day for an additional two to five days. In a specific, non-limiting example, 2 mL EOM613 is administered twice a day for five days, followed by once a day for an additional 5 days. In another non-limiting example, 2 mL EOM613 is administered once a day for ten days.

In some embodiments, the therapeutically effective amount of the EOM613 is administered orally, parenterally, topically, intranasally, by inhalation, or systemically. Parenteral administration includes subcutaneous injections, intravenous, intramuscular, intraperitoneal, intrapleural, intrasternal injection, or infusion techniques. In a non-limiting example, the therapeutically effective amount of EOM613 is administered parenterally by subcutaneous injection. In some embodiments, the EOM613 is administered directly to the lungs, for example, by inhalation or by endotracheal tube. By way of example, one method of administration to the lungs is by inhalation through the use of a nebulizer or inhaler. For example, the EOM613 is formulated as an aerosol or particulate and drawn into the lungs using a standard nebulizer. The preferred administration route may vary, for example, with the condition and age of the recipient.

In some embodiments including administration by inhalation, EOM613 is conveniently delivered in the form of an aerosol spray presentation from pressurized packs or a nebulizer, with the use of a suitable propellant, for instance, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges for use in an inhaler or insufflator can be formulated containing the EOM613 and any appropriate additives as needed.

In some embodiments of the methods provided herein, at least one additional therapeutic agent is administered to the subject, such as an anti-viral agent, anti-bacterial agent, anti-parasite agent, or an anti-inflammatory agent. In non-limiting examples, the additional agent is remdesivir, hydroxychloroquine, azithromycin, dexamethasone, chloroquine phospate, ivermectin, alpha-interferon (for example, interferon α2b), beta-interferon, gamma-interferon, lambda-interferon, ritonavir, arabinol, or an anti-Tumor Necrosis Factor (TNF)-α antibody, such as adalimumab.

In some embodiments, in addition to the EOM613, oxygen is administered to the subject with a small amount of continuous positive airway pressure (“CPAP”). Additionally, intravenous fluids may be administered to stabilize the blood sugar, blood salts, and blood pressure.

### IV. Pharmaceutical Formulations That Include EOM613

EOM613 may be administered alone or as part of a pharmaceutical formulation. It may also be administered at about the same time as one or more other pharmaceuticals independently administered to the subject. If EOM613 is administered as part of a pharmaceutical formulation, the formulations of the present invention comprise at least one administered ingredient, i.e., EOM613, together with one or more pharmaceutically acceptable carriers, and optionally one or more additional medicaments. The carrier(s) must be pharmaceutically acceptable in the sense of being compatible with the other ingredients of the formulation and not deleterious to the recipient (e.g., suitable for in vivo use, in the subject).

EOM613 in a pharmaceutical formulation is sterile. Pharmaceutical formulations may be presented in unit-dose or multi-dose containers, e.g., sealed ampules and vials. Preferred unit dosage formulations are those containing a daily dose or unit, daily sub-dose, or an appropriate fraction of the administered ingredient.

An EOM613 pharmaceutical formulation can be in the form of a lyophilized solid for reconstitution in a normal saline solution, liquid or gas (aerosol). Pharmaceutical formulations comprising EOM613 can be formulated so as to allow EOM613 or other compound to become bioavailable upon administration of the pharmaceutical composition to a subject. Pharmaceutical formulations can take the form of one or more dosage units. For example, a container of EOM613 in aerosol form can hold a plurality of dosage units. A syringe containing a unit dose of EOM613 is also provided.

For oral administration, the pharmaceutical formulations can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical formulations can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrates (e.g., potato starch or sodium starch glycolate); or wetting agents (e.g., sodium lauryl sulphate). The tablets can be coated by methods well-known in the art.

Preparations for oral administration can be suitably formulated to give controlled release of the active compound. For buccal administration, the compositions can take the form of tablets or lozenges formulated in conventional manner.

For administration by inhalation, a pharmaceutical formulation can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit can be determined by providing a valve to deliver a metered amount. Capsules and cartridges for use in an inhaler or insufflator can be formulated containing a powder mix of EOM613 and a suitable powder base, such as lactose or starch.

A pharmaceutical formulation can be formulated for parenteral administration by injection, for example by bolus injection or continuous infusion. Formulations for injection can be presented in unit dosage form, e.g., in ampoules or in multi-dose containers, with an added preservative. The formulations can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical formulations can be formulated in compositions such as creams, lotions, gels, ophthalmic drops, ointments, solutions, suspensions, shampoos, or other forms known to one of skill in the art and described in, for example, Remington's Pharmaceutical Sciences, 16th and 18th eds., Mack Publishing, Easton Pa. (1980 & 1990), and Introduction to Pharmaceutical Dosage Forms, 4th ed., Lea & Febiger, Philadelphia (1985). Actual methods for preparing pharmaceutical compositions are known or apparent to those skilled in the art and are described in detail in, for example, Remington's Pharmaceutical Sciences, 16th and 18th eds., Mack Publishing, Easton Pa. (1980 & 1990).

In addition to the pharmaceutical formulations described previously, EOM613 can also be formulated as a depot preparation. Such long acting formulations can be administered by implantation (e.g., subcutaneously or intramuscularly) or by intramuscular injection. In such examples, the compounds can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

### EXAMPLES

The following examples are provided to illustrate particular features of certain embodiments, but the scope of the claims should not be limited to those features exemplified.

### Example 1

### Production of EOM613

This example illustrates an embodiment of the process of making EOM613.

Suspend about 35.0 g of casein, about 17.1 g of beef peptone, about 22.0 g of nucleic acid (yeast RNA), about 3.25 g bovine serum albumin in about 2.5 liters of water for injection USP at about 3 to 7° C. in a suitable container and gently stir until all the ingredients have been properly wet. Carefully add while stirring about 16.5 g of sodium hydroxide (reagent grade ACS) and continue stirring until sodium hydroxide completely dissolved. Autoclave at about 9 lbs. pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C.

The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/100 ml, the final volume is approximately 5 liters. The pH is then adjusted with either concentrated HCl (reagent grade ACS) or 1.0 normal NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclaved for final sterilization at 240° F. and 14-16 pounds pressure for about 30 minutes. Following the sterilization cycle, the ampules with EOM613 are cooled and washed.

All quantities are subject to plus or minus 15% variation for pH, volume, and analytical adjustments.

### Example 2

### Treatment of SARS-COV-2 Infection With EOM613

This example provides a treatment protocol for a patient with SARS-COV-2 infection using the EOM613 composition described in Example 1.

A patient with SARS-COV-2 infection is selected for treatment. The patient can have an active SARS-COV-2 infection without symptoms (as a prophylactic treatment against development of severe symptoms) or with symptoms, including severe symptoms such as myocardial disease, kidney dysfunction, clotting disorder, encephalitis, severe fatigue or multisystem inflammatory syndrome. The patient is administered 2 ml EOM613 subcutaneously twice per day for 3 days followed by 1 ml EOM613 subcutaneously twice per day for 7 days.

Typically, the patient is assessed before and during the treatment period. Physical examination includes assessment of consciousness, orientation to time and place, body temperature, pulse, blood pressure, heart rate, EKG, respiratory rate, chest x-ray. Clinical laboratory data to be collected include CBC, CRP, ESR, procalcitonin, SMA-18, troponin, natriuretic peptides, blood gases, urinalysis. If the patient does not respond to the initial 10-day treatment protocol, the treatment may be repeated.

### Example 3

### Treatment of Severe SARS-COV-2 Infection With EOM613

A 91-year old male subject with anemia, tested positive for SARS-COV-2 infection by RT-PCR. The subject presented fever, general malaise and weakness, and overt clinical signs of pulmonary discomfort and lung congestion. The subject was entirely bedridden, debilitated and immobile, unable even to move by himself in bed, requiring others' assistance. The subject had an oxygenation SpO2 percentage level in the 80s and required supplemental oxygen. Ten days after his symptoms had first appeared, a lung ultrasound was performed and showed extreme pulmonary congestion. Twelve days after the initial symptomatic presentation, initial blood test data were obtained that were used as the starting point for further analysis following EOM treatment. Two days subsequent to the initial blood test data, the therapy with EOM 613 was initiated, and the course of the patient's progress was followed subsequent to initiation of treatment for an additional 24 days.

For treatment, the subject received a 2 mL dose administered by subcutaneous injection twice daily for the first 2 days, followed by a 2 mL dose administered once daily by subcutaneous injection for three additional days. The subject received no concomitant antiviral or immunomodulatory agent or corticosteroid treatment; only over-the counter antipyretics to reduce his fever.

The day after initiation of EOM613 treatment, the patient first opened his eyes. By day 2, he was able to turn himself in bed without assistance. The subject's lung congestion was also noted to be resolving. By day 4 after initiation of EOM613 treatment the subject had resolved his pulmonary symptoms, was ambulatory, and had diminished fever or was non-febrile. By day 15, the patient was well enough to be able to independently get out of bed, walk and go to the bathroom unaided by himself.

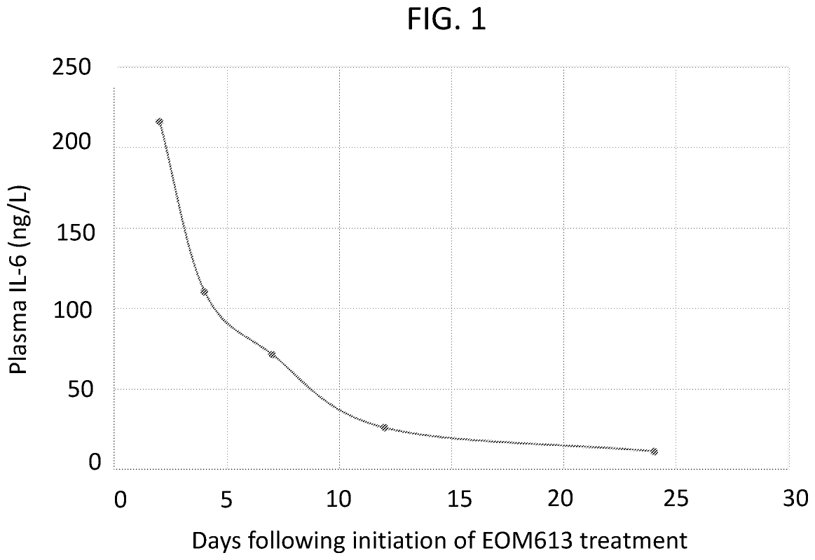

The subject's plasma IL-6 levels, which had risen to 216.1 ng/L as of day 2 post-initiation of treatment, were reduced to 110.3 ng/L by day 4 post-initiation of treatment, then fell to 71.3 ng/L by day 7 post-initiation of treatment, and then further declined to 25.7 ng/L on day 12 and 11 ng/L on day 24 post-treatment (FIG. 1). The diminution of plasma IL-6 levels observed with EOM613 treatment was in concert with and concomitant with the resolution of the observed inflammatory and pulmonary symptoms of COVID-19 infection.

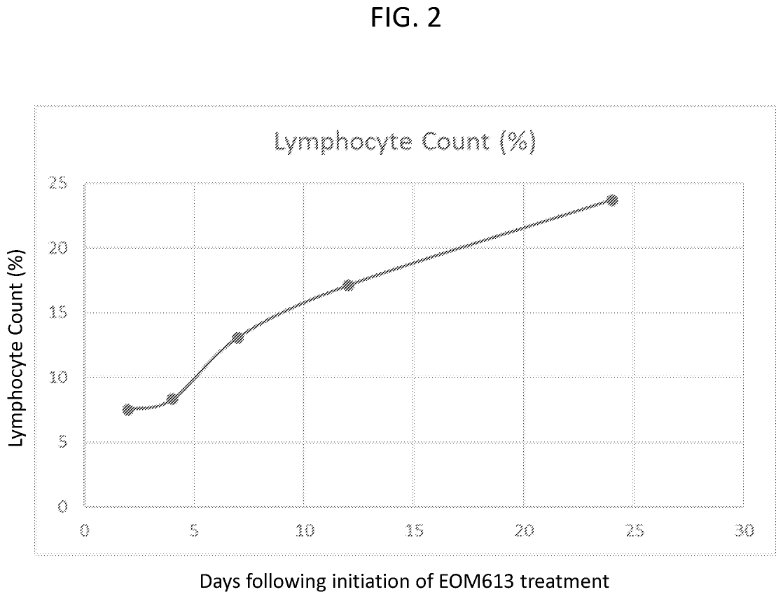

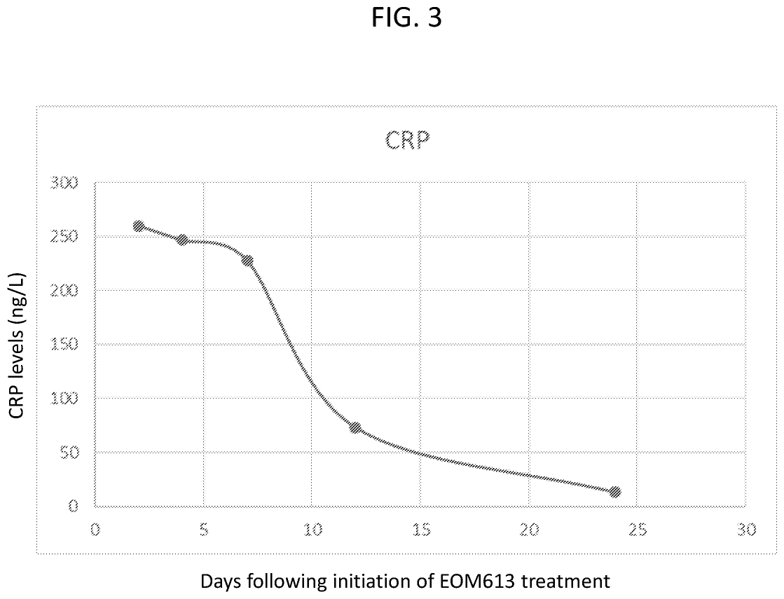

Improvement in the subject's COVID-19 related lymphocytopenia was also seen with the EOM613 treatment (FIG. 2). On Day 2 post-initiation of treatment the subject had presented with a very low lymphocyte count of 7.5% in his differential blood cell count; at 7 days post-initiation of treatment the count had risen to 13.1%. By Day 12, the subject's lymphocyte percentage count had risen to 17.1%, and by day 24 it had further rebounded to a value of 23.7%. Plasma C-reactive protein (CRP) levels also diminished from a high of 260.1 ng/L to 73.3 ng/L twelve days post-initiation treatment, and then further reduced to 13 ng/L 24 days post-initiation of EOM613 treatment (FIG. 3). The subject went from a SpO2 percentage oxygenation level in the 80s on day one and requiring supplemental oxygen, to breathing on his own with an SpO2 level of over 95% by day 24.

### Example 4

### Treatment of Severe SARS-COV-2 Infection in an Obese Patient With EOM613

A 31-year old male patient with a comorbidity of obesity was hospitalized with severe symptoms of COVID-19 infection, confirmed by RT-PCR testing. The patient presented fever, difficulty breathing, respiratory distress, body ache and recent loss of taste and smell. He was hospitalized in an ICU setting, and quickly progressed to requiring mechanical ventilation, with a score of 6 on the WHO Ordinal Scale for Clinical Improvement. For reference, the 8-point WHO Ordinal Scale for Clinical Improvement is provided below in Table 2. At this point the patient began treatment with EOM613 for a period of 10 days, receiving 2 mL twice a day the first five days and 2 mL once daily the remaining five days. The Table 1 shows the patient's record of improvement.

TABLE 1
Improvement in WHO score and biochemical
parameters over the course of treatment.

| | Day 1 | Day 2 | Day 5 | Day 8 | Day 11 | Day 28 |
|---|---|---|---|---|---|---|
| WHO Score | 6 | 5 | 4 | 4 | 3 | 0 |
| CRP (mg/mL) | 42 | 155 | 145 | 38.6 | 16.8 | N/A |
| Troponin-I | 71 | 31.1 | 13.3 | 5 | 6.5 | N/A |
| (ng/mL) | | | | | | |
| Procalcitonin | 0.110 | 3.84 | 0.110 | 0.05 | 0.05 | 0.05 |
| (ng/mL) | | | | | | |

TABLE 2
The 8-point WHO Ordinal Scale for Clinical Improvement.

| Patient State | Descriptor | Score |
|---|---|---|
| Uninfected | No clinical or virological evidence of infection | 0 |
| Ambulatory | No limitation of activities | 1 |
| | Limitation of activities | 2 |
| Hospitalized - | Hospitalized, no oxygen therapy | 3 |
| Mild Disease | Oxygen by mask or nasal prongs | 4 |
| Hospitalized - | Non-invasive ventilation or high-flow oxygen | 5 |
| Severe Disease | Intubation and mechanical ventilation | 6 |
| | Ventilation + additional organ support - | 7 |
| | pressor, RRT, ECMO | |
| Dead | Death | 8 |

By Day 2 the patient was off mechanical ventilation, improving to a WHO score of 5, requiring high flow oxygen. The patient showed further improvement upon evaluation on Day 5 with a WHO score of 4 and by Day 11 had improvement to a score of 3 (still hospitalized, but no need for supplemental oxygen). The patient was discharged from hospital on Day 16. On a Day 28 follow-up was entirely disease free and had resumed normal activity, with an assigned WHO score of zero.

Biochemical parameters tested with the progression of EOM613 treatment on Days 1, 2, 5, 8 and 11 showed that the C-Reactive Protein (CRP) transiently rose on Day 2 to a high value of 155 mg/mL, but then declined steadily over the course of the treatment period, having dropped to 16.8 mg/mL by Day 11. The elevated troponin-I level concomitantly dropped from a high of 71 ng/mL on Day 1 to a value of 5 ng/mL on Day 8 and 6.5 ng/ml on Day 11 with treatment progression. Procalcitonin blood levels dropped from a value of 0.110ng/ml to 0.05 ng/mL on Day 8 and beyond, which was within a normal range. EOM613 also produced a drop of pro-inflammatory CRP over the course of the treatment after an initial transient rise in this patient. It also produced a therapeutically positive steady drop in procalcitonin and troponin-I, elevations of which are regarded as prognostic biomarkers of disease progression and worsening prognostic outcomes in COVID-19 patients

### Example 5

### Treatment of SARS-COV-2 Infection in an Asthmatic Patient With EOM613

A 31-year old female patient with a past history of severe bronchial asthma presented with symptoms of COVID-19 including cough, fever and chills, shortness of breath, and recent loss of smell and taste. The patient required the use of supplemental oxygen, and was assigned a WHO Ordinal Scale for Clinical Improvement of 4 upon admission (see, Table 2). She presented with normal troponin-I levels, but displayed an elevated D-dimer level of 4.5 mg/mL (normal <0.5 mg/mL). She commenced treatment with EOM613 at a dose of 2 mL given subcutaneously once daily for 10 days. On Day 2 her WHO score had improved to a 3 and by Day 5 to a score of 1. The patient was discharged from hospital with a disease-free outcome and no limitation of activities by Day 4.

Since the patient had an elevated D-dimer level at the time of hospital admission, she was monitored for this biomarker in blood tests. On Day 2 her D-dimer level a reading of 1.81 mg/mL and by Day 5 it had further dropped to 1.35 mg/mL.

It will be apparent that the precise details of the methods or compositions described may be varied or modified without departing from the spirit of the described embodiments. We claim all such modifications and variations that fall within the scope and spirit of the claims below.

1. A method of treating a subject with severe acute respiratory syndrome coronavirus 2 (SARS-COV-2) infection, comprising administering a therapeutically effective amount of EOM613 to the subject.

2. The method of claim 1, further comprising selecting a subject having SARS-COV-2 infection for treatment.

3. The method of claim 1, wherein the subject has or is at risk of Coronavirus disease 2019 (COVID-19).

4. The method of claim 1, wherein treating the subject reduces at least one of the following symptoms of the SARS-COV-2 infection: respiratory distress, lung inflammation, hypoxia, myocarditis, renal disease, blood clotting, encephalitis, pneumonia, profound weakness, lymphocytopenia, cytokine storm syndrome, and multisystem inflammatory syndrome.

5. The method of claim 1, comprising administering to the subject an amount of EOM613 effective to inhibit SARS-COV-2 induced multisystem inflammatory syndrome.

6. The method of claim 5, wherein inhibiting the SARS-COV-2 induced multisystem inflammatory syndrome reduces organ damage to the heart, kidneys, and/or lungs.

7. The method of claim 5, wherein the subject is a child.

8. The method of claim 1, wherein the subject has one or more of the following underlying medical conditions: heart disease, cancer, chronic obstructive pulmonary disease, type 2 diabetes, type 1 diabetes, obesity, chronic kidney disease, sickle cell disease, asthma, liver disease, chronic lung disease, high blood pressure, or a suppressed immune system due to medical treatment, infection with a pathogen other than SARS-COV-2, or an autoimmune disorder.

9. The method of claim 1, wherein the subject is a human.

10. The method of claim 1, wherein the subject is at least 75 years old.

11. The method of claim 1, wherein the subject has lymphocytopenia due to the SARS-COV-2 infection and wherein administering the therapeutically effective amount of EOM613 to the subject increases lymphocyte count in the subject to a range of 20-40% of white blood cells.

12. The method of claim 1, wherein the subject has an elevated level of one or more of D-dimer, troponin, C-reactive protein, procalcitonin, or cytokine, and wherein administering the therapeutically effective amount of EOM613 to the subject decreases the elevated level to a non-detectable or a normal level.

13. The method of claim 12, wherein the subject has an elevated level of one or more of D-dimer, troponin-I, C-reactive protein, or procalcitonin.

14. The method of claim 1, wherein administering the therapeutically effective amount of EOM613 to the subject comprises administering about 0.5 microliters to about 100 microliters EOM613 per kilogram of body weight per day to the subject.

15. The method of claim 14, wherein administering the therapeutically effective amount of EOM613 to the subject comprises administering about 2.5 microliters to about 40 microliters of EOM613 per kilogram of body weight per day to the subject.

16. The method of claim 14, wherein administering the therapeutically effective amount of EOM613 to the subject comprises administering about 10 microliters to about 25 microliters of EOM613 per kilogram of body weight per day to the subject.

17. The method of claim 1, wherein administering the therapeutically effective amount of EOM613 to the subject comprises administering about 1 to about 2 ml EOM613 twice per day to the subject.

18. The method of claim 17, wherein administering the therapeutically effective amount of EOM613 to the subject comprises administering about 2 ml EOM613 twice per day to the subject for two to five days.

19. The method of claim 18, wherein the administering about 2 ml EOM613 twice per day to the subject for two to five days is subsequently followed by administering about 1 to about 2 ml EOM613 once per day to the subject for an additional two to five days.

20. The method of claim 17, wherein administering the therapeutically effective amount of EOM613 to the subject comprises administering about 2 ml EOM613 twice per day to the subject for three days, and then administering about 1 ml EOM613 twice per day to the subject for seven days.

21. The method of claim 1, wherein administering the therapeutically effective amount of EOM613 to the subject comprises administering about 2 ml EOM613 once per day to the subject for about ten days.

22. The method of claim 1, wherein the therapeutically effective amount of EOM613 is administered parenterally, topically, by inhalation, or systemically.

23. The method of claim 22, wherein the therapeutically effective amount of EOM613 is administered by inhalation using a nebulizer or an inhaler.

24. The method of claim 22, wherein the therapeutically effective amount of EOM613 is administered parenterally by subcutaneous injection.

25. The method of claim 1, further comprising administering to the subject at least one additional anti-COVID-19 agent.

26. The method of claim 25, wherein the additional anti-COVID-19 agent is an antiviral agent.

27. The method of claim 1, wherein the subject has a score of 5 or higher on the World Health Organization (WHO) Ordinal Scale for Clinical Improvement prior to initiation of treatment.

28. The method of claim 27, wherein the subject has a score of 6 or higher on the WHO Ordinal Scale for Clinical Improvement prior to initiation of treatment.

29. The method of claim 25, wherein the additional anti-COVID-19 agent is one or more of remdesivir, hydroxychloroquine, azithromycin, dexamethasone, chloroquine phosphate, ivermectin, alpha-interferon, beta-interferon, gamma-interferon, ritonavir, or arabinol.

30. A method of treating a subject with lymphocytopenia, comprising administering an amount of EOM613 effective to treat the lymphocytopenia to the subject.
